(19) United States
(12) Patent Application Publication
(10) Pub. No.: US 2005/0070601 A1
(43) Pub. Date: Mar. 31, 2005

(54) Psychostimulant Effects of Forskolin Including Anorexia

(75) Inventors: Derek Wayne Cornelius, Cape Girardeau, MO (US); Gary Lee Haynes, Scott City, MO (US)

Correspondence Address: CREATIVE COMPOUNDS, LLC, P.O. BOX 4011, SCOTT CITY, MO, 63780, US

(73) Assignee: CREATIVE COMPOUNDS, LLC, Scott City, MO (US)

(21) Appl. No.: 10/605,406

(22) Filed: Sep. 29, 2003

(51) Int. Cl. A61K 31/343

(52) U.S. Cl. 514/469

(57) ABSTRACT

Forskolin is disclosed to have novel effects on humans such as stimulating various aspects of the metabolism so that the overall effect is one of perceived increased energy levels and improved mental alertness. As part of this psychostimulation, forskolin shows a profound effect to reduce a human's appetite for food. Forskolin can be safely used either as a drug or as a food or dietary supplement to decrease a human's food intake. It can likewise be safely used as a mental stimulant so that the person experiences a heightened sense of energy levels as well as mental alertness.

### BACKGROUND OF INVENTION

### REFERENCES CITED

U.S. Pat. No. 5,804,596 September, 1998 Majeed

### OTHER REFERENCES

Huang Z Y., et al. “Obesity and the central nervous system regulation.” Sheng Li Ke Xuejin Zhan 32 (1):45-51, 2001
Chaki S., et al. “Cocaine- and amphetamine-regulated transcript peptide produces anxiety-like behavior in rodents.” Eur. J. Pharmacol. 464 (1):49-54, 2003
Miller D K., et al. “Sensitization of anorexia and locomotion induced by chronic administration of ephedrine in rats.” Life Sci. 65 (5):501-11, 1999
Nencini P., et al. Sensitization to kappa opioid mechanisms associated with tolerance to the anorectic effects of cathinone.”J. Pharmacol. Exp. Ther. 245 (1):147-54, 1988

### DETAILED DESCRIPTION

For thousands of years, mankind has been searching for a safe and effective mental stimulant. Many have been found, but, so far, none have proven to be both safe and effective over an extended period of time. Caffeine may be the safest and is initially quite effective. However, it rather quickly loses its ability to provide a noticeable increase in mental stimulation. After a period of several weeks users tend to use the caffeine merely to feel “normal” as its ability to induce a heightened sense of energy is vastly diminished. Even worse, caffeine has a tendency to habituate its users so that when its use is intermittently broken a person will have a decreased sense of energy and alertness. Even with these drawbacks, caffeine is widely used and accepted because of its overall safety profile.

Besides caffeine, many other psychostimulants exist but they have a much greater risk to benefit ratio. Items such as the various amphetamines, cocaine, cathinone, and the ephedrine derivatives are quite effective but they present an unacceptable risk to benefit ratio. The amphetamines, cocaine, and cathinone become highly addictive with increased usage. Cocaine, cathinone and the amphetamines are classified as controlled substances by most countries worldwide and have few or no medical usages. Ephedrine and its derivates are not addictive or habituating by most indicators but they have come under increased scrutiny because of their association with negative cardiovascular phenomenon such as heart attack and stroke.

The inventors quickly realized that there is a relationship between psychostimulants and their use as appetite suppressants. All of the above mentioned stimulants have been used or are currently being used as anorectic agents. Caffeine is currently being widely used in a plethora of weight loss dietary formulas to enhance the appetite suppressing properties of other agents. Ephedrine and its derivates, cocaine, cathinone and amphetamines are excellent appetite suppressants. It is to be noted that they are also the most effective and most potent of the psychostimulants.

An improvement over the above agents is needed. A psychostimulant is thus highly desired which is safe and effective for increasing a human's perceived energy levels and alertness. It would also be highly beneficial if this psychostimulant could be used as an effective anorectic agent.

Forskolin is a diterpine compound that can be naturally extracted from Coleus forskohli or synthetically manufactured. Several synthetic methods have been developed but due to the complex nature of the synthetic techniques, the cost becomes prohibitive. Thus, all commercially available forskolin being sold is naturally extracted from Coleus forskohli. Because there is no difference between naturally occurring forskolin or synthetically derived forskolin, either can be successfully used as per this invention. Currently, standardized Coleus forskohli extracts are available which range from 1% to 99% forskolin with 10%, 20% and 40% extracts being most common.

Forskolin in the form of Coleus forskohli and its extracts have been traditionally used for centuries in the human diet for such things as asthma and heart disease. It is also known in the public domain that forskolin is beneficial for raising thyroid hormone levels, decreasing high blood pressure, and improving skin conditions such as eczema and psoriasis.

In 1998, a patent was issued to Sabinsa Corp. for the use of forskolin for increasing the proportion of lean body mass in a human.

Daily dosages for the above uses of forskolin are usually less than 60 mg per day. In U.S. Pat. No. 5,804,596 issued to Sabinsa Corp., the maximum daily dosage for increasing the proportion of lean body mass in a human is 60 mg per day.

While experimenting with forskolin in much higher daily dosages, the inventors discovered new and as yet undiscovered uses for forskolin and Coleus forskohli and its extracts. The novel effects became quite evident and noticeable upon administering 150 mg per day of forskolin in the form of a 20% Coleus forskohli extract. Test subjects noticed a definite psychostimulant effect such as an increase in perceived energy levels, wakefulness and alertness. Upon further investigation, it was found that forskolin's psychostimulant properties are about as strong as caffeine. To the inventors surprise, it was also found that forskolin did not cause any untoward central nervous system stimulating effects such as feelings of jitteriness or nervousness or increased blood pressure, heart rate or respiratory rate. The dosages of forskolin that precipitate the best psychostimlant properties seem to be above 100 mg but can be below this level for some highly sensitive people.

Because nearly all psychostimulants have been or are being used as extremely effective appetite suppressants, the inventors decided to test the hypothesis that forskolin would be an effective anorectic agent. The inventors quickly realized that forskolin in dosages that cause psychostimulation also creates in humans a potent appetite suppressant effect. This anorectic effect is dose dependent and works increasingly until the dosage of 500 mg per day of forskolin. Most tested individuals noticed the appetite suppression effects above 75 mg per day of forskolin; however, a few individuals noticed slight effects down to 30 mg per day.

This is first time that psychostimulation and appetite suppression effects have been attributed to forskolin. As with most other psychostimulants, the appetite suppression effects of forskolin seem to be inexorably linked to its psychostimulation properties. We have not determined whether the mechanism is similar to other psychostimulants or a new, as yet to be discovered, novel mechanism. What makes forskolin superior to previous technology is its safety profile as well as lack of negative side effects. This usage of forskolin is extremely novel and a definite improvement over prior technology.

Human dosaging for the novel use of forskolin as per this invention ranges from 30 mg to 500 mg per day. Our experience though shows that the most effective dosage is between 60 mg and 300 mg per day. Due to reasonable cost and benefit factors though, the most preferred dosage will be between 75 mg and 150 mg per day. Although the forskolin of this invention can be dosed as little as once per day for a desired effect, we have found that it is best to divide the dosage into two or three equal amounts given eight to twelve hours apart or alternatively about 30-60 minutes before meals. This helps to ensure steady blood values and an overall more powerful, consistent effect. Effective dosaging may be in the form of tablets, capsule, sachets, effervescent powder or tablets, softgels, liquid or other oral delivery system known in the art.

### EXAMPLE 1

Forskolin in the form of a Coleus forskohli extract standardized for 40% forskolin was administered to 5 individuals to measure the psychostimulant effects of forskolin. The forskolin was administered as capsules at a dosage of 100 mg twice daily both morning and night for three consecutive days. The forskolin was administered one hour after waking and one hour before bedtime. Subjects were asked to evaluate their level of wakefulness/alertness on a scale of 1 to 10 (10 being the greatest) both before administration and one hour after administration. The average results from the three days are as follows:

TABLE 1

| Subject | MBA | MAA | EBA | EAA |
|---|---|---|---|---|
| One | 3 | 5 | 6 | 7 |
| Two | 2 | 7 | 5 | 9 |
| Three | 7 | 8 | 7 | 5 |
| Four | 7 | 10 | 7 | 10 |
| Five | 3 | 5 | 6 | 8 |

MBA = morning, before administration
MAA = morning, after administration
EBA = evening, before administration
EAA = evening, after administration

As can be seen from the above data, forskolin in the form of a standardized 40% extract exerts a profound psychostimulant effect. All of the subjects perceived an increase in their level of wakefulness/alertness both in the morning and in the evening. These times were chosen due to the fact that most people have a greater degree of sleepiness or a greater degree of having problems with mental alertness during these times of the day.

### EXAMPLE 2

Forskolin in the form of a Coleus forskohli extract standardized for 40% forskolin was administered to 3 overweight individuals who claimed to have a problem controlling their appetite. The forskolin was administered as capsules at a dosage of 100 mg sixty minutes before meals. The forskolin was administered three times per day for five days. Subjects were asked to evaluate the level of their appetite from a scale of 1 to 10 (10 being the greatest). The average results from the five days are as follows:

TABLE 2

| Subject | BB | AB | BL | AL | BD | AD |
|---|---|---|---|---|---|---|
| One | 6 | 5 | 9 | 7 | 9 | 8 |
| Two | 8 | 3 | 10 | 3 | 10 | 3 |
| Three | 4 | 4 | 8 | 5 | 7 | 5 |

BB = Breakfast, Before Administration
AB = Breakfast, After Administration
BL = Lunch, Before Administration
AL = Lunch, After Administration
BD = Dinner, Before Administration
AD = Dinner, After Administration

As can be seen from the above results, forskolin at the novel dosage levels of this invention caused a profound decrease in each of the subject's appetite. Some of the subjects even reported that they felt no desire to eat within 30 minutes after administration even though before administration they felt “extremely hungry”.

### EXAMPLE 3

Forskolin in the form of a Coleus forskohli extract standardized for 40% forskolin was administered to 5 individuals to measure any negative effects from forskolin such as nervousness or an increase in blood pressure or heart rate. The forskolin was administered as capsules at a dosage of 200 mg once per day. Subjects were analyzed one hour after administration. Testing occurred for 3 consecutive days. The results are as follows:

TABLE 3

| Subject | Nervousness | Increased Heart Rate | Increased Blood Pressure |
|---|---|---|---|
| One | None | None | None |
| Two | None | None | None |
| Three | None | None | None |

As can be seen from the above data, unlike other psychostimulants such as caffeine, ephedrine, and amphetamines, forskolin did not have an adverse affect on any of the tested parameters. For this reason, forskolin is a vast improvement over previously used psychostimulants such as caffeine, ephedrine or amphetamines.

1. Administering an effective amount of forskolin to a human for the novel purpose of psychostimulation without adverse side effects.

2. Psychostimulation according to claim 1 which consists of increased mental alertness and wakefulness.

3. Side effects according to claim 1 which consists of increased heart rate, feelings of nervousness, increased blood pressure and addictiveness.

4. Forskolin according to claim 1 which is obtained through synthetic means or through extraction from Coleus forskohli.

5. An effective amount of forskolin according to claim 1 which is between 30 mg and 500 mg per day.

6. An effective amount of forskolin according to claim 5 which is most preferable between 75 mg and 150 mg per day.

7. Forskolin according to claim 1 that is administered perorally as tablets, hard or soft gelatin capsules, sachets, or as a powder, including effervescent powder, or liquid.

8. Administering an effective amount of forskolin to a human for the novel purpose of reducing food intake and appetite for food without adverse side effects.

9. Adverse side effects according to claim 8 which consists of increased heart rate, feelings of nervousness, increased blood pressure and addictiveness.

10. Forskolin according to claim 8 which is obtained through synthetic means or through extraction from Coleus forskohli.

11. An effective amount of forskolin according to claim 8 which is between 30 and 500 mg per day.

12. An effective amount of forskolin according to claim 11 which is most preferable between 75 and 150 mg per day.

13. Forskolin according to claim 8 that is administered perorally as tablets, hard or soft gelatin capsules, sachets, or as a powder, including effervescent powder, or liquid.
